(19) United States
(12) Patent Application Publication
(10) Pub. No.: US 2026/0209305 A1
(43) Pub. Date: Jul. 23, 2026

(54) THERAPEUTIC EXPLOITATION OF STING CHANNEL ACTIVITY

(71) Applicants: The Broad Institute Inc., Cambridge, MA (US); The General Hospital Corporation, Boston, MA (US); Massachusetts Institute of Technology, Cambridge, MA (US)

(72) Inventors: Nir Hacohen, Brookline, MA (US); Bingxu Liu, Cambridge, MA (US); Rebecca Carlson, Boston, MA (US); Matteo Gentili, Cambridge, MA (US)

(21) Appl. No.: 19/130,761

(22) PCT Filed: Nov. 15, 2023
(86) PCT No.: PCT/US23/79800

§ 371 Date: May 16, 2025

Related U.S. Application Data
(60) Provisional application No. 63/425,828, filed on Nov. 16, 2022.

(51) Int. Cl. C07K 14/705; A61K 38/00

(52) U.S. Cl. CPC C07K 14/705; A61K 38/00

(57) ABSTRACT

In certain aspects, provided herein are mutant stimulator of interferon genes (STING) polypeptides, compositions thereof and methods of using the same, wherein the mutation is within the dimerization interface of the STING, and wherein the mutation reduces the ability of the mutant STING polypeptide to mediate protein leakage across a membrane compared to a wild-type STING polypeptide.

### RELATED APPLICATIONS

This application claims the benefit of priority to U.S. Provisional Application No. 63/425,828 filed Nov. 16, 2022.

### STATEMENT OF GOVERNMENT SUPPORT

This invention was made with government support under Grant Nos. AI133524 and AI158495 awarded by the National Institutes of Health. The government has certain rights in the invention.

### BACKGROUND

The first-line of defense against infection is provided by the innate immune system. A major innate immunity pathway for detecting viruses or bacteria is mediated by cyclic GMP-AMP (cGAMP) synthase (cGAS). cGAS serves as a DNA sensor by directly binding to pathogen DNA in the cytosol and generates the second messenger cGAMP. STimulator of INterferon Genes (STING) is a conserved mammalian receptor that is essential for sensing cyclic dinucleotides derived directly from bacteria (Burdette et al. 2011) or synthesized by cGAMP synthase (cGAS) from cytosolic DNA (Sun et al. 2013; Wu et al. 2013). Upon binding to agonist, STING undergoes a conformational change and translocates from the ER to the Golgi and endosomes, where it carries out multiple biological functions, including interferon induction (Ishikawa and Barber 2008), autophagy (Gui et al. 2019), and inflammasome activation (Gaidt et al. 2017).

### SUMMARY

Provided herein are mutant of STimulator of INterferon Genes (STING) polypeptides. In certain aspects, the present disclosure provides a mutant STING polypeptide comprising a mutation within its dimerization interface. In some embodiments, the mutation reduces the ability of the mutant STING polypeptide to mediate protein leakage across a membrane compared to a wild-type STING polypeptide of SEQ ID NO. 1. In some embodiments, the mutation reduces the ability of the mutant STING polypeptide to mediate protein leakage across a membrane. In some embodiments, the mutation allows the mutant peptide to induce interferon induction but reduces its ability to mediate inflammasome activation and/or autophagy compared to a wild-type STING polypeptide of SEQ ID NO. 1.

### BRIEF DESCRIPTION OF THE DRAWINGS

FIG. 1A-D depicts (A) workflow for the genome-wide CRISPR screen. (B) Genome-wide CRISPR screen resulted in highly significant scores for v-ATPase and non-canonical autophagy components but no known ion transporter (GO: 0015075, ion transmembrane transporter activity) scored strongly (C) STING abundance and localization in WT and TMEM192-STING-LBD cells, scale bar 10 μm. (D) p-STING and LC3B lipidation immunoblot for 293T cells expressing WT STING, STING AQQA, TMEM192-STING-LBD.

FIG. 2A-C depicts (A) proposed pore for chicken cGAMP-induced STING but not the apo conformation; key parameters shown below. (B) Images of BJ1 cells at 10 and 50 minutes, post diABZI stimulation with or without C53. Scale bar 10 μm. (C) Quantification of experiment in (B), *** indicates p<0.0001 at 50 minutes by a Tukey HSD test performed after two-way ANOVA.

FIG. 3A-G depicts (A) visualization of stably expressed RFP-LC3 and STING-HA in FIP200 KO 293T cells upon cGAMP stimulation with or without C53 co-treatment. Scale bar 10 μm (B) Quantification of experiment in (A). *** indicates p<0.001. (C) Western blot for p-STING and LC3B lipidation in BJ1 cells with or without co-treatment with C53 upon cGAMP or MSA2 stimulation. (D) Amino acid positions targeted in the mutagenesis screen, transmembrane domain of human apo STING (PDB: 6NT5) shown. (E) Mutagenesis screen workflow. (F) Mutagenesis screen results show depletion or enrichment of mutated STING constructs (p-value, y-axis; fold-change construct abundance, x-axis) comparing RFP-LC3-high vs low in FIP200 KO 293T cells. (G) RFP-LC3 fluorescence intensity within the pSTING+ or pSTING-populations in FIP200 KO 293T cells overexpressing STING WT or H50R. Quantification shown in bar graph. **** indicates p<0.0001 using a student t-test.

FIG. 4A-D depicts (A) STING translocation and NLRP3 puncta in untreated, diABZI-treated cells with or without C53 co-treatment and nigericin-treated 293T cells expressing STING-HA and NLRP3-mNeongreen. Scale bar 5 μm (B) Proportion of cells with NLRP3 puncta and per-cell mean NLRP3 puncta intensity based on (A). One-way ANOVA followed by Tukey's HSD. * indicates p<0.05, *** indicates p<0.001. (C) Experimental workflow for inflammasome induction in primary human monocytes; (D) Western blot from stimulated human monocytes showing induction of processed IL 1b upon cGAMP or diABZI stimulation in the absence or presence of C53 or the NLRP3 inhibitor MCC950.

FIG. 5A-B depicts (A) replicate log-fold-change correlations for genome-wide CRISPR screen (B) Replicate-log 10 (FDR adjusted p value) correlations for genome-wide CRISPR screen.

FIG. 6A-D depicts (A) radius, charge and hydropathy of the proposed channel area. (B) (C) Representative images of images from lysosomal pH tracking experiment quantified in (B). (D) Images of full time-course for selected images in FIG. 2B, scale bar 10 μm.

FIG. 7A-D depicts (A) proportion of cells expressing LC3 puncta among cells positive for STING upon cGAMP and diABZI treatment with or without C53 co-treatment. (B) (C) Replicate correlations for targeted STING saturated mutagenesis screen. (D) pSTING activation measured by FACS is similar in WT and STING H50R cells.

### DETAILED DESCRIPTION

### General

Human STING is a 379-amino acid (aa) protein (encoded by the 8-exon TMEM173 gene) and has several functional domains. The N-terminal region (aa 1-139) contains four transmembrane (TM) domains responsible for membrane anchoring. The C-terminal region of STING (aa 139-379) protrudes into the cytosol and harbors the cyclic dinucleotides (CDNs)-binding domain (CBD, aa 155-340). Amino acids 340-379 form STING's C-terminal tail (CTT), which functions as a platform for interaction with TBK1 and IRF3.

| (SEQ ID NO. 1) | |
|---|---|
| 1 | mphsslhpsi pcprghgaqk aalvllsacl vtlwglgepp ehtlrylvlh laslqlglll |
| 61 | ngvcslaeel rhihsryrgs ywrtvraclg cplrrgalll lsiyfyyslp navgppftwm |
| 121 | lallglsqal nillglkgla paeisavcek gnfnvahgla wsyyigylrl ilpelqarir |
| 181 | tynqhynnll rgavsqrlyi llpldcgvpd nlsmadpnir fldklpqqtg dhagikdrvy |
| 241 | snsiyellen gqragtcvle yatplqtlfa msqysqagfs redrleqakl fcrtledila |
| 301 | dapesqnncr liayqepadd ssfslsqevl rhlrqeekee vtvgslktsa vpststmsqe |
| 361 | pellisgmek plplrtdfs |

STING mediates the cellular response to DNA and bacteria by activating three distinct processes: interferon secretion, autophagy, and inflammasome activation. However, the mechanisms underlying autophagy and inflammasome activation are not well-understood. Disclosed herein is STING acting as a channel with a pore formed by its dimerization interface. Without being bound by any particular mechanism or theory, STING activation induced a pH increase in the acidic Golgi/endosome compartment, which can be blocked by a pore-binding small molecule, C53. Provided herein are mutations (e.g., H50R) inside the pore area which strongly inhibit STING-induced autophagy and STING-induced NLRP3 inflammasome activation but not STING phosphorylation. The role of STING as a channel strengthens the concept of ion leakage as a danger signal that triggers autophagy and inflammasome activation to defend against pathogens. STING was shown to induce non-canonical autophagy that requires the conjugation of ATG8 to single membranes (CASM) (Xu et al. 2022; Fischer et al. 2020). CASM can be triggered by proton leakage from membrane damage (Xu et al. 2019), specific channels such as influenza M2 (Ulferts et al. 2021) or proton ionophores (Hooper et al. 2022). It was investigated whether proton leakage could be involved in STING-induced CASM. Furthermore, some studies have shown that proton leakage can lead to NLRP3 activation (Ichinohe, Pang, and Iwasaki 2010).

Provided herein are nucleic acids (e.g., mRNAs) encoding mutants of STING, particularly mutants wherein the mutation (at least one mutation) is located within the dimerization interface. For example, such mutations may be located within the transmembrane helices TM1-TM4. In some embodiments, the at least one mutation is located in TM2 and/or TM4. Nucleic acids encoding mutant forms of STING, e.g., mutant human STING isoforms with reduced, or otherwise lacking, induction of autophagy and/or inflammasome activation are provided herein. The amino acid residue numbering for mutant human STING polypeptides used herein corresponds to that used for the 379 amino acid residue wild type human STING available in the art as Genbank Accession Number NP 938023.

The compositions and methods of the present disclosure rely, at least in part, on the uncoupling of STING-induced interferon induction from STING-induced autophagy and/or inflammasome activation. For example, and without limitation, compositions comprising the STING polypeptides disclosed herein can be used to induce interferon production without STING-induced autophagy and/or inflammasome activation.

In some aspects, provided herein are mutants of STING polypeptide comprising a mutation within the dimerization interface. In some such embodiments, the mutation reduces the ability of the mutant STING polypeptide to mediate protein leakage across a membrane compared to a wild-type STING polypeptide of SEQ ID NO. 1. In other embodiments, the mutation reduces the ability of the mutant STING polypeptide to mediate inflammasome activation compared to a wild-type STING polypeptide of SEQ ID NO. 1. In certain embodiments, the mutation reduces the ability of the mutant STING polypeptide to mediate protein leakage across a membrane and/or reduces the ability of the mutant STING polypeptide to mediate inflammasome activation compared to a wild-type STING polypeptide of SEQ ID NO. 1. In some embodiments, the mutation is within a transmembrane helix of the mutant STING polypeptide. For example, the mutation is within TM2 or TM4. In some preferred embodiments, the mutation is within TM2. In some such embodiments, the mutation is a substitution mutation.

The mutations contemplated herein may be located at a position of the mutant STING polypeptide corresponding to a position between amino acids Y46 and K137 of SEQ ID NO. 1. In some such embodiments, the mutation is located at a position of the mutant STING polypeptide corresponding to Y46, H50, S53, L54, S127, Q128, N131, or K137 of SEQ ID NO. 1. In some preferred embodiments, the mutation is a H50R mutation of the STING polypeptide, e.g., corresponding to the wild-type STING polypeptide of SEQ ID NO. 1.

In certain aspects, provided herein are nucleic acids encoding the mutant STING polypeptides disclosed herein. In other aspects, provided herein are vectors comprising the nucleic acids contemplated herein. In some such embodiments, the vector is selected from nanoparticles, adenovirus vectors, adeno-associated virus (AAV) vectors, retrovirus vectors, picorna virus vectors, liposomes, cationic lipid systems, and protein/nucleic acid complexes.

In other aspects, provided here are cells comprising the nucleic acids disclosed herein. In certain aspects, provided here are cells comprising the vectors disclosed herein. In further aspects, provided herein are cells expressing the mutant STING polypeptides disclosed herein. For example and without limitation, the cell is an endothelial cell, epithelial cell, neuronal cell, or haematopoietic cell. In some such embodiments, the haematopoietic cell is an immune cell selected from a lymphocyte, a monocyte, a macrophage, a dendritic cell, a mast cell, a neutrophil, a basophil, or an eosinophil. In certain embodiments, the immune cell is a lymphocyte selected from a an αβT cell, γδT cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, or a regulatory T cell.

In certain embodiments, the cells contemplated herein are cells of the central nervous system (CNS) or peripheral nervous system (PNS). In other embodiments, the cell is a cell of the bone marrow.

In some embodiments, the nucleic acids contemplated herein may refer to a polymeric form of nucleotides or nucleosides of any length, such as deoxyribonucleotides or ribonucleotides, or analogs thereof. Nucleic acids may have any three-dimensional structure, and may perform any function. The following are non-limiting examples of Nucleic acids: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides/polynucleosides, branched polynucleotides/polynucleosides, plasmids, vectors, isolated DNA of any sequence, isolated RNA of any sequence, nucleic acid probes, and primers. A nucleic acid may comprise modified nucleotides/nuclosides, such as methylated nucleotides/nuclosides and nucleotide/nucloside analogs. If present, modifications to the polynucleotide/polynucloside structure may be imparted before or after assembly of the polymer. A polynucleotide/polynucloside may be further modified, such as by conjugation with a labeling component.

Aspects include therapeutic compositions comprising the mRNAs disclosed herein. In some embodiments, the therapeutic composition comprises a vector selected from nanoparticles, adenovirus vectors, adeno-associated virus (AAV) vectors, retrovirus vectors, picorna virus vectors, liposomes, cationic lipid systems, and protein/nucleic acid complexes. For example and without limitation, the therapeutic composition comprising an mRNA may be formulated in a lipid nanoparticle (LNP). In some embodiments of the therapeutic composition, one or more of the uridine nucleosides in the mRNA are N1-methylpseudouridine. In some such embodiments, all of the uridine nucleosides in the mRNA are N1-methylpseudouridine. In some embodiments of the therapeutic composition, the LNP comprises an ionizable lipid, a structural lipid, a phospholipid, a sterol, a PEG-modified lipid, or any combination thereof.

In some aspects, provided herein are methods of treating a cancer in a subject, the method comprising administering an effective amount of a therapeutic composition contemplated herein. In some embodiments, the cancer is selected from: hepatocellular carcinoma, lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

In some such embodiments, the therapeutic composition is administered intrapleurally, intravenously, subcutaneously, intranodally, intratumorally, intrathecally, intraperitoneally, intracranially, or by direct administration to an organ.

In certain embodiments, the method further comprises administering to the subject a cancer immunotherapy. The cancer immunotherapy may comprise administration of an immune checkpoint inhibitor. In some such embodiments, the immune checkpoint inhibitor comprises an antibody or antigen-binding fragment thereof specific for PD-1, PD-L1, or CTLA4. In certain embodiments, the cancer immunotherapy comprises administration of a CAR-T cell or a CAR-NK cell.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, peptide or nucleic acid described herein.

The phrases “therapeutically-effective amount” and “effective amount” as used herein means the amount of an agent, which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### Nucleic Acids and Vectors

Nucleic acids and vectors disclosed herein include polynucleotides and polynucleotide vectors encoding the disclosed STING mutants that allow expression in the disclosed cells.

Nucleic acid sequences encoding the disclosed STING mutants can be obtained using recombinant methods known in the art. Alternatively, the gene of interest, STING, can be produced synthetically, rather than cloned.

In some embodiments, a nucleic acid of the disclosure may be modified in a coding region (e.g., an open reading frame of an mRNA encoding a polypeptide). In other embodiments, nucleic acid may be modified in regions besides a coding region, such as, a 5′-UTR and/or a 3′-UTR 5 of an mRNA, wherein either or both may independently contain one or more different nucleoside modifications. In such embodiments, modifications may also be present in the coding region.

Examples of nucleoside modifications and combinations thereof that may be present in mRNAs disclosed herein include, but are not limited to, those described in PCT Patent Application Publications: WO2012045075, WO2014081507, WO2014093924, WO2014164253, WO2014159813, each of which are incorporated herein in their entirety.

In some embodiments, the mRNAs of the disclosure can include a combination of modifications to the sugar, the nucleobase, and/or the internucleoside linkage. These combinations can include any one or more modifications described herein. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

Expression of nucleic acids encoding STING mutants is typically achieved by operably linking a nucleic acid encoding the STING mutant polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acids can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses and AAVs provide a convenient platform for gene delivery systems. A selected gene can be inserted into a vector and packaged in viral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-la (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a STING polypeptide disclosed herein or portions thereof, the expression vector to be introduced into a cell can also contain either a selectable marker gene or a reporter gene or both to facilitate identification and selection of expressing cells from the population of cells sought to be transfected or infected through viral vectors. The selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes may be used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the nucleic acid has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/nucleic acid or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances, which may be naturally occurring or synthetic lipids. For example, lipids include the fatty droplets that naturally occur in the cytoplasm as well as the class of compounds, which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

In some embodiments the nucleic acids of the disclosure may be formulated in nanoparticles or other delivery vehicles, e.g., to protect them from degradation when delivered to a subject. Illustrative nanoparticles are described in Panyam, J. & Labhasetwar, V. Adv. Drug Deliv. Rev. 55, 329-347 (2003) and Peer, D. et al. Nature Nanotech. 2, 751-760 (2007), each of which are incorporated herein by reference in their entirety. In certain embodiments, an mRNA of the disclosure is encapsulated within a nanoparticle. In particular embodiments, a nanoparticle is a particle having at least one dimension (e.g., a diameter) less than or equal to 1000 nM, less than or equal to 500 nM or less than or equal to 100 nM. In particular embodiments, a nanoparticle includes lipid. Lipid nanoparticles (LNPs) include, but are not limited to, liposomes and micelles. Any of a number of lipids may be present, including cationic and/or ionizable lipids, anionic lipids, neutral lipids, amphipathic lipids, PEGylated lipids, and/or structural lipids. Such lipids can be used alone or in combination. In certain embodiments, a lipid nanoparticle comprises one or more nucleic acids, e.g., mRNAs, described herein. In certain embodiments, it is desirable to target a nanoparticle, e.g., a lipid nanoparticle, of the disclosure using a targeting moiety that is specific to a cell type and/or tissue type. In some embodiments, a nanoparticle may be targeted to a particular cell, tissue, and/or organ using a targeting moiety. In particular embodiments, a nanoparticle comprises one or more mRNA described herein and a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′)2 fragments), single domain antibodies, camelid antibodies and fragments thereof, human antibodies and fragments thereof, monoclonal antibodies, and multispecific antibodies (e.g., bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42(5): 439-62, 2003 and Abra et al., J. Liposome Res. 12:1-3, 2002. For example, the lipid nanoparticle may include a targeting moiety that targets the lipid nanoparticle to a cell including, but not limited to, hepatocytes, colon cells, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells). In particular embodiments, the targeting moiety targets the lipid nanoparticle to a hepatocyte. In other embodiments, the targeting moiety targets the lipid nanoparticle to a colon cell. In some embodiments, the targeting moiety targets the lipid nanoparticle to a liver cancer cell (e.g., a hepatocellular carcinoma cell) or a colorectal cancer cell (e.g., a primary tumor or a metastasis).

In certain embodiments, lipid nanoparticles (LNPs) comprise lipids including an ionizable lipid, a structural lipid, a phospholipid, and one or more mRNAs. Each of the LNPs described herein may be used as a formulation for the mRNA described herein. In one embodiment, a lipid nanoparticle comprises an ionizable lipid, a structural lipid, a phospholipid, a PEG-modified lipid and one or more mRNAs. In some embodiments, the LNP comprises an ionizable lipid, a PEG-modified lipid, a sterol and a phospholipid. In some embodiments, the LNP has a molar ratio of about 20-60% ionizable lipid:about 5-25% phospholipid:about 25-55% sterol; and about 0.5-15% PEG-modified lipid. In some embodiments, the LNP comprises a molar ratio of about 50% ionizable lipid, about 1.5% PEG-modified lipid, about 38.5% cholesterol and about 10% phospholipid. In some embodiments, the LNP comprises a molar ratio of about 55% ionizable lipid, about 2.5% PEG lipid, about 32.5% cholesterol and about 10% phospholipid. In some embodiments, the ionizable lipid is an ionizable amino or cationic lipid and the neutral lipid is a phospholipid, and the sterol is a cholesterol. In some embodiments, the LNP has a molar ratio of 50:38.5:10:1.5 of ionizable lipid:cholesterol:DSPC (1,2-dioctadecanoyl-sn-glycero-3-phosphocholine):PEG-DMG.

### Compositions

In some aspects, provided herein is a composition (e.g., a pharmaceutical composition, such as a therapeutic or vaccine composition), containing . . . , formulated together with a pharmaceutically acceptable carrier, as well as methods of administering such pharmaceutical compositions.

In some embodiments, the polypeptides or compositions provided herein are used as an adjuvant. As used herein, the term “adjuvant” broadly refers to an agent that affects an immunological or physiological response in a patient or subject. For example and without limitation, when used as an adjuvant the polypeptides or compositions provided herein may increase the presence of an antigen over time or to an area of interest like a tumor, facilitate absorption of a presented antigen, activate macrophages and lymphocytes, and/or support the production of cytokines. By changing an immune response, the adjuvant might permit a smaller dose of an immune interacting agent to increase the effectiveness or safety of a particular dose of the immune interacting agent. For example, the adjuvant might prevent T cell exhaustion and thus increase the effectiveness or safety of a particular immune interacting agent.

Compositions contemplated herein may be administered intrapleurally, intravenously, subcutaneously, intranodally, intratumorally, intrathecally, intraperitoneally, intracranially, or by direct administration to an organ. Said compositions may comprise one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

### Therapeutic Methods

In certain embodiments, provided herein are methods of treating a subject comprising administering to the subject a therapeutic or vaccine composition provided herein.

In some embodiments, provided herein is a method of treating a viral or bacterial infection in a subject. In some embodiments, the subject treated is immunocompromised. For example, in some embodiments, the subject has a T cell deficiency. In some embodiments, the subject has leukemia, lymphoma or multiple myeloma. In some embodiments, the subject is infected with HIV and/or has AIDS. In some embodiments, the subject has undergone a tissue, organ and/or bone marrow transplant. In some embodiments, the subject is being administered immunosuppressive drugs. In some embodiments, the subject has undergone and/or is undergoing a chemotherapy. In some embodiments, the subject has undergone and/or is undergoing radiation therapy.

In some embodiments, the subject is also administered an anti-viral drug that inhibits viral replication. For example, in some embodiments, the subject is administered ganciclovir, valganciclovir, foscarnet, cidofovir, acyclovir, formivirsen, maribavir, BAY 38-4766 or GW275175X.

In some embodiments, the subject has cancer. In some embodiments, the methods described herein may be used to treat any cancerous or pre-cancerous tumor. In some embodiments, the cancer includes a solid tumor. Cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the compositions and methods provided herein can be used to treat an autoimmune disease. Examples of autoimmune diseases include, for example, glomerular nephritis, arthritis, dilated cardiomyopathy-like disease, ulceous colitis, Sjogren syndrome, Crohn disease, systemic erythematodes, chronic rheumatoid arthritis, juvenile rheumatoid arthritis, Still's disease, multiple sclerosis, psoriasis, allergic contact dermatitis, polymyositis, pachyderma, periarteritis nodosa, rheumatic fever, vitiligo vulgaris, Behcet disease, Hashimoto disease, Addison disease, dermatomyositis, myasthenia gravis, Reiter syndrome, Graves' disease, anaemia perniciosa, sterility disease, pemphigus, autoimmune thrombopenic purpura, autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus erythematosus, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, lymphopenia, mixed connective tissue disease, pemphigoid, pemphigus vulgaris, pernicious anemia, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, type I diabetes, ulcerative colitis and Wegener's granulomatosis.

In some embodiments, the methods provided herein are used to treat multiple sclerosis (MS). In some embodiments, the MS is relapsing-remitting MS, secondary progressive MS, primary progressive MS or progressively relapsing MS.

In certain embodiments, the methods provided herein are used to treat rheumatoid arthritis, systemic lupus erythematosus and/or Sjögren's syndrome.

In some embodiments, the methods provided herein are used to treat inflammatory bowel diseases (IBDs). For example, in certain embodiments the methods provided herein are used to treat Crohn's disease (regional bowel disease, e.g., inactive and active forms), celiac disease (e.g., inactive or active forms) and/or ulcerative colitis (e.g., inactive and active forms). In some embodiments, the methods provided herein are used to treat irritable bowel syndrome, microscopic colitis, lymphocytic-plasmocytic enteritis, coeliac disease, collagenous colitis, lymphocytic colitis, eosinophilic enterocolitis, indeterminate colitis, infectious colitis (viral, bacterial or protozoan, e.g. amoebic colitis) (e.g., clostridium dificile colitis), pseudomembranous colitis (necrotizing colitis), ischemic inflammatory bowel disease, Behcet's disease, sarcoidosis, scleroderma, IBD-associated dysplasia, dysplasia associated masses or lesions, and/or primary sclerosing cholangitis.

In some embodiments, the methods provided herein are used to treat a disease, disorder, condition, and/or illness associated with inflammation can include, but not limited to, septic shock, obesity-related inflammation, Parkinson's Disease, Crohn's Disease, Alzheimer's Disease (AD), cardiovascular disease (CVD), inflammatory bowel disease (IBD), chronic obstructive pulmonary disease, an allergic reaction, an autoimmune disease, blood inflammation, joint inflammation, arthritis, asthma, ulcerative colitis, hepatitis, psoriasis, atopic dermatitis, pemphigus, glomerulonephritis, atherosclerosis, sarcoidosis, rheumatoid arthritis, psoriatic arthritis, ankylosing spondylitis, Wegner's syndrome, Goodpasture's syndrome, giant cell arteritis, polyarteritis nodosa, idiopathic pulmonary fibrosis, acute lung injury, post-influenza pneumonia, SARS, tuberculosis, malaria, sepsis, cerebral malaria, Chagas disease, schistosomiasis, bacteria and viral meningitis, cystic fibrosis, multiple sclerosis, encephalomyelitis, sickle cell anemia, pancreatitis, transplantation, systemic lupus erythematosis, autoimmune diabetes, thyroiditis, and radiation pneumonitis, respiratory inflammation, or pulmonary inflammation.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

In some embodiments, the methods provided herein further comprise treating the identified subject using a therapeutic method provided herein (e.g., by administering to the subject a pharmaceutical composition provided herein).

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection. In certain embodiments, the disclosed compositions are administered to a patient in conjunction with (e.g., before, simultaneously or following) any number of relevant treatment modalities, including but not limited to thalidomide, dexamethasone, bortezomib, and lenalidomide. In further embodiments, the compositions may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the said compositions are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In other embodiments, the compositions disclosed herein are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells disclosed herein. In additional embodiments, expanded cells are administered before or following surgery.

### EXAMPLES

### Example 1: Materials and Methods

FACS based genome-wide CRISPR screen and mutagenesis screen: FIP200 KO 293T cells were generated by transfecting 293T cells with pXPR_023 (Addgene #52961), encoding Cas9 and an sgRNA against FIP200 and selecting cells with 2 μg/ml puromycin (Thermo Fisher Scientific #A1113803) for two days. FIP200 KO 293T cells were then transduced with pTRIP-PGK-Hygro-P2A-RFP-LC3, treated with 33 μg/ml hygromycin (Invivogen, #ant-hg-1) for 3 days, and then sorted to get a homogenous RFP+ population.

Genome-wide CRISPR screen: RFP-LC3 FIP200 KO 293T cells were transduced with pTRIP-PGK-Blast-P2A-STING-HA and select with 20 μg/ml blasticidin (Thermo Fisher Scientific #A1113903). For screening, 200M RFP-LC3+ STING-HA+ FIP200 KO 293T cells were then transduced with Cas9-sgRNA all-in-one Brunello library at MOI=0.4 and selected with 2 μg/ml puromycin for 2 days. 8 days after transduction, 200M transduced cells were treated with 1 μM diABZI (Invivogen, #tlrl-diabzi) for 2.5 hr, and then permeabilized with 1× perm buffer (PBS with 0.05% saponin and 0.1% glycine) for 4 min. Permed cells are then washed with 1×PBS, and fix, perm, and stained using BD Cytofix/Cytoperm kit (#554714) anti-HA Alexa647 (Biolegend, #682404) 1:200 dilution

Mutagenesis screen: RFP-LC3 FIP200 KO 293T cells were infected with saturated mutagenesis library lentivirus at MOI=0.2, and selected with 20 μg/ml blasticidin for 2 days. 150M transduced cells were treated with diABZI for 1.5 hr, and then permeabilized with 1× perm buffer (PBS with 0.05% saponin and 0.1% glycine) for 4 min. Permed cells are then washed with 1×PBS, and fix, perm, and stained using BD Cytofix/Cytoperm kit and anti-pSTING Alexa647 (CST, #43499) at 1:100 dilution.

Organelle pH Measurements and Image Acquisition: hTERT-immortalized BJ1 cells (ATCC CRL-2522) were plated in 24-well glass-bottom plates (Greiner Bio-One). After 24 hours, cells were stained for 30 minutes at 37° C. with 5 μM Lysosensor (Thermo Fisher Scientific cat #L7535), 2 g/ml Hoechst (Thermo Fisher Scientific, cat. #62249), and 1 μM Bodipy TR Ceramide (Thermo Fisher Scientific cat #B34400) for Golgi labeling or 100 nM Lysotracker Deep Red (Thermo Fisher Scientific cat #L12492) for endolysosome labeling in Phenol-free DMEM medium with 10% FBS and 1% Pen-Strep. Cells were then washed five times with Phenol-free DMEM medium and stimulated with 1 μM DIABZI for 1 hr with or without the addition of 10 μM C53. All images were acquired using a Ti-2 Eclipse inverted epifluorescence microscope (Nikon) with automated XYZ stage control, hardware autofocus, and an Okolab cage incubator. An LED light engine (Lumencor CELESTA Light Engine) was used for fluorescence illumination and all hardware was controlled using NIS elements software. Images were acquired using a 40×0.95 NA CFI Plan Apo 2 objective (Nikon) with the following filters: DAPI (405 nm laser, Chroma Multi LED set #89402), Lysosensor (445 nm laser, Chroma LED set #96372 with no excitation filter), Bodipy (546 nm laser, emission 615/24 nm, dichroic 565 nm), Lysotracker (637 nm laser, emission 680/42 nm, dichroic 660 nm).

Autophagy Induction and Image Acquisition: RFP-LC3 and STING-HA-expressing FIP200 KO 293T cells were seeded on Fibronectin bovine plasma coated coverslips at 0.2 million cells/well density in a 24 well plate the night before stimulation. Cells were then stimulated with 20 ug/ml cGAMP (Invivogen, #tlrl-nacga23-1)+5 ug/ml or 1 μM DIABZI (Invivogen, #tlrl-diabzi) with or without the addition of 10 μM C53 (Cayman, #37354) for 1 hour. Cells were then fixed with 2% Paraformaldehyde (Electron Microscopy Sciences) in PHEM buffer (Electron Microscopy Sciences) for 30 minutes at 37° C., washed three times with PBS and quenched with freshly prepared 0.1M Glycine for 10 minutes. Coverslips were then permeabilized and blocked with 1× perm/stain buffer (10% goat serum (Thermo Fisher) in PBS, 0.5% BSA (Seracare), 0.05% Saponin from quillaja barka (Sigma)) for 30 minutes. Coverslips were then stained with anti-HA (Millipore, #11867423001) for 1 hour at room temperature in 1× perm/stain buffer, washed 5 times, and then stained with Alexa 647 anti-rat IgG (H+L) (Thermo, A-21247) in 1× perm/stain buffer for 1 hour. Coverslips were then washed 5 times, mounted with Fuoromont-G, with DAPI (Thermo Fisher) and dried at 37° C. for one hour. Images were acquired on Olympus. Images were acquired on an Olympus IX83 using an Olympus UApo 40× objective controlled by Fluoview software.

NLRP3 Stimulation and Image Acquisition: HEK293T cells expressing NLRP3-mNeonGreen and STING-HA were plated in 24-well glass-bottom plates (Greiner Bio-One) and, after 24 hours, stimulated with 2 μM nigericin or 1 μM DIABZI with or without the addition of 10 μM C53 for 1 hour. Cells were then fixed with 2% Paraformaldehyde (Electron Microscopy Sciences) in PHEM buffer (Electron Microscopy Sciences) for 30 minutes at 37° C., washed three times with PBS and quenched with freshly prepared 0.1M Glycine for 10 minutes. Cells were then permeabilized and blocked with 1× perm/stain buffer (10% goat serum (Thermo Fisher) in PBS, 0.5% BSA (Seracare), 0.05% Saponin from quillaja barka (Sigma)) for 30 minutes. Cells were then stained with anti-HA (Millipore, #11867423001) for 1 hour at room temperature in 1× perm/stain buffer, washed 5 times, and then stained with Alexa 647 anti-rat IgG (H+L) (Thermo, A-21247) in 1× perm/stain buffer for 1 hour, and imaged in 2×SSC with 200 ng/mL DAPI. All images were acquired using a Ti-2 Eclipse inverted epifluorescence microscope (Nikon) with automated XYZ stage control and hardware autofocus. An LED light engine (Lumencor CELESTA Light Engine) was used for fluorescence illumination and all hardware was controlled using NIS elements software. Images were acquired using a 40×0.95 NA CFI Plan Apo λ objective (Nikon) with the following filters: DAPI (405 nm laser, Chroma Multi LED set #89402), mNeon Green (473 nm laser, Chroma LED set #96372), AF647 (637 nm laser, emission 680/42 nm, dichroic 660 nm).

Western Blotting: BJ1 cells were seeded at 0.15 million cells/well density in a 24 well plate the night before stimulation. Cells were then stimulated with 2 μM Nigericin (Invivogen, #tlrl-nig), 20 ug/ml cGAMP (Invivogen, #tlrl-nacga23-1)+5 ug/ml or 40 μM MSA2 (Invivogen, #tlrl-diabzi) with or without the addition of 10 μM C53 (Cayman, #37354) for 1 hour. Cells were then harvested and lysed in 100 ul of 1×SDS sample buffer (Boston Bio, #BP111NR).

Monocyte Experiments: CD14+ monocytes were isolated from peripheral adult human blood as previously described. CD14+ monocytes were cultured in RPMI (Gibco) supplemented with 10% FBS (VWR), 1× GlutaMax (Thermo Fisher), 50 ug/ml Gentamicin (Thermo Fisher) and 1× Penicillin/Streptomycin (Corning)

FACS Screen Analysis: Guide RNA abundances were extracted from FASTQ files using poolq 3.3.2 with fixed row and barcode policies. Resulting log-abundances were then subtracted between sorting bins from each experimental condition and analyzed using the Broad Genetic Perturbation Platform screen analysis tool (portals.broadinstitute.org/gpp/public/analysis-tools/crispr-gene-scoring) using a hypergeometric analysis.

Image Analysis: Analysis was performed using code adapted from (Feldman et al. 2022). Cells were segmented by thresholding the DAPI signal to identify nuclei and expanding the resulting regions using the seeded watershed method. Cells were segmented based on background in the Lysosensor, LC3B-RFP, and NLRP3 mNeonGreen channels. For the live-cell imaging and NLRP3 stimulation experiments, background was subtracted using the rolling ball algorithm. Puncta or Golgi structures enhanced using a Laplacian of Gaussian filter with sigma=12 (live-cell), sigma=8 (NLRP3), or sigma=7 (LC3B).

### Example 2: STING-Induced Autophagy Genes

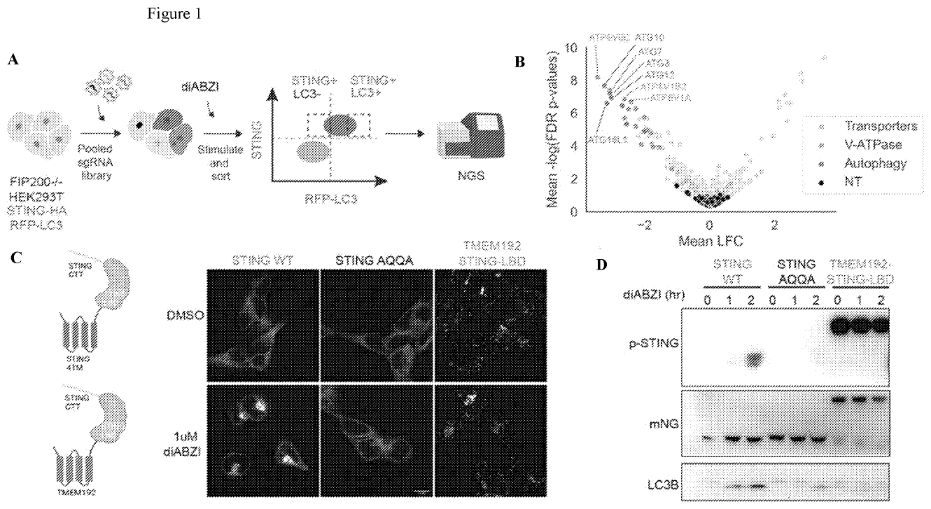

Genes that are essential for mediating STING-induced autophagy were systematically identify, with a special interest in identifying potential transporters responsible for ion leakage. LC3B lipidation induced by STING activation, a process conserved from Nematostella to human STING (Gui et al. 2019), does not require essential components of canonical autophagy such as VPS34 or FIP200/RB1CC1 (Fischer et al. 2020). A genome-wide CRISPR fluorescence-activated cell sorting (FACS) screen was carried out using HEK293T cells expressing RFP-LC3 and STING-HA with FIP200 knocked out in order to reduce background signal from basal autophagy. Following transduction with the sgRNA library, cells were stimulated with diABZI to induce STING activation and STING-HA+ cells were sorted into LC3− and LC3+ bins (FIG. 1A) to specifically identify genes that altered CASM activation without affecting STING activation. While the screen identified critical CASM regulators, including ATG16L1, V-ATPase components, and other non-canonical autophagy factors (FIG. 1B) and showed strong technical reproducibility (FIG. 5A). However V-ATPase assembly was shown to be required as a scaffold for recruiting ATG16L1 rather than for its proton pumping function (Hooper et al. 2022; Xu et al. 2022, 2019), and no other known transporters scored as top hits in our screen. The STING itself may have mediated Golgi/endosomes proton leakage critical for subsequent assembly of V-ATPase and recruitment of ATG16L1 to initiate CASM (Xu et al. 2019). In order to assess this, whether the STING ligand-binding domain (LBD), which has been proposed to recruit LC3 through its LR motifs (Gui et al. 2019; Liu et al. 2019), could induce autophagy upon translocation to Golgi/endosomes was first tested. Autophagy induction was tested in 293T cells by WT STING, a STING oligomerization-deficient mutant (A277Q/Q273A STING, termed AQQA), and an endolysosome-localized STING fusion protein (the endolysosomal protein TMEM192 fused with the STING LBD) upon agonist stimulation. While the AQQA mutant impaired LC3B lipidation, TMEM192-STING-LBD did not induce autophagy despite its endolysosomal localization and strong induction of STING phosphorylation (FIG. 1C, D). Thus, STING translocation with its transmembrane domain is critical for induction of CASM.

### Example 3: STING as a Channel for Proton Release

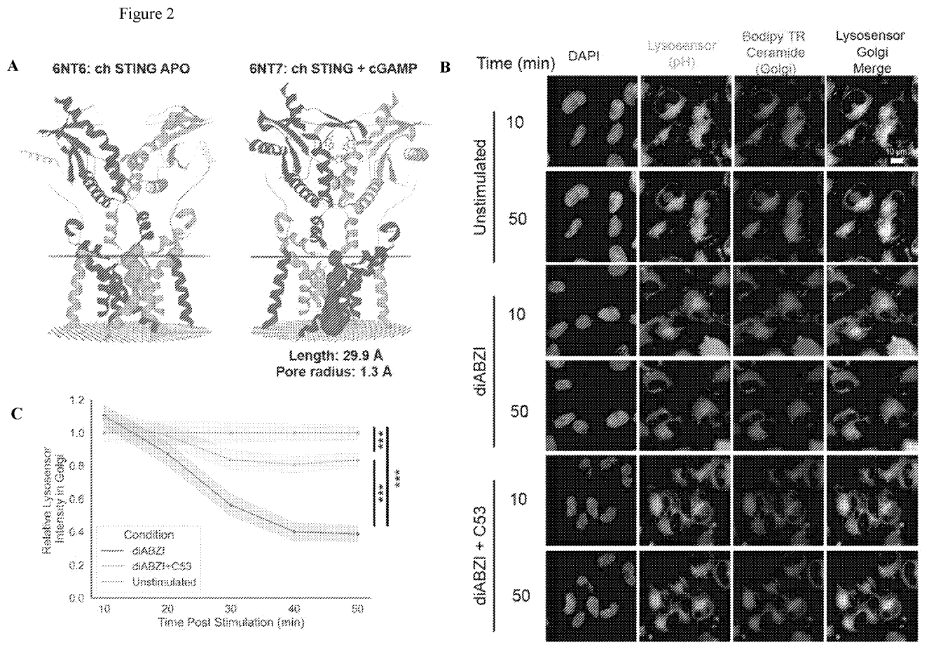

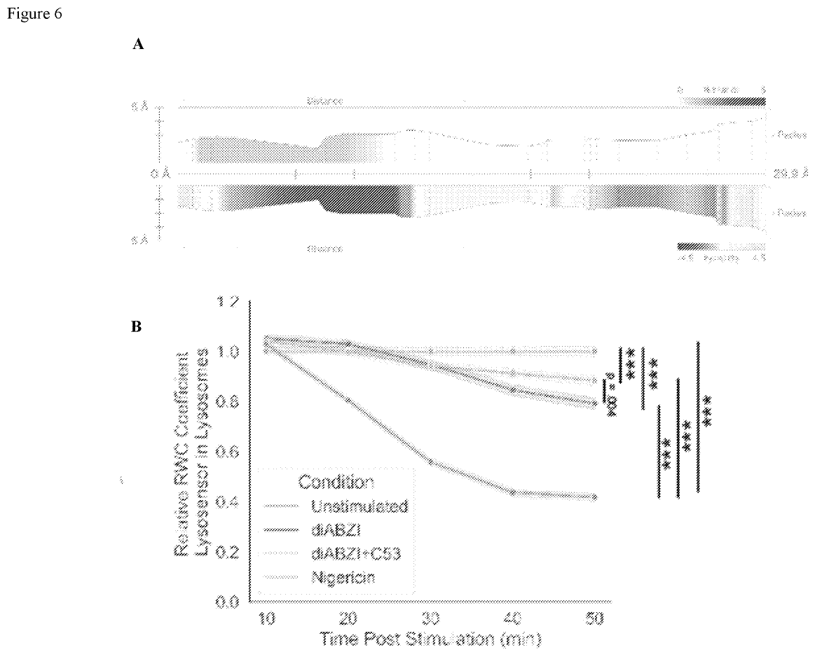

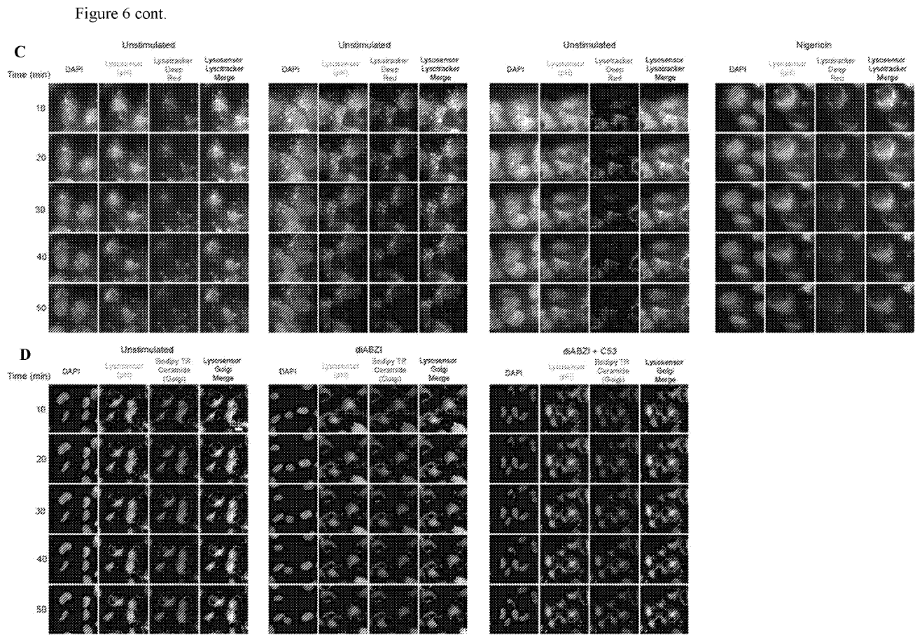

Given the necessity of both the STING transmembrane domain and its translocation for autophagy induction, it was investigated whether the entire STING protein could cause ion leakage by either inducing membrane damage resulting in a secondary ion imbalance or by directly acting as an ion channel. Previous research has shown that STING translocation induced LC3 lipidation without inducing Galectin-3 puncta (Xu et al. 2022), suggesting that STING does not result in membrane damage. The possibility that STING directly behaves as a channel for proton release into the cytosol upon translocation to the Golgi was assessed, the Golgi being an acidic compartment (Linders et al. 2022). Given that the function of STING-induced autophagy is evolutionarily conserved (Gui et al. 2019), the structure of chicken STING should provide a general mechanistic understanding to determine whether STING could function as an ion channel. Using MOLEonline, a tool for automated location and characterization of channels in macromolecules (Pravda et al. 2018), it was found that apo chicken STING possesses only a central cavity in the dimerization interface, while in the cGAMP-bound conformation, STING has a pore that spans through the whole lipid bilayer (1.3 Å bottleneck radius, 29.9 Å length) (FIG. 2A). In order to test whether this pore could lead to a change in pH upon STING activation, human fibroblasts were stimulated with diABZI and a significant increase in pH was observed, denoted by decreased fluorescence of the Lyosensor Green dye, within the Golgi apparatus (FIGS. 2B and C, FIG. 6A), whereas fluorescence within the endolysosomal compartment (FIG. 6B, C) was only modestly altered. The recently discovered agonistic compound 53 (C53) (Lu et al. 2022) binds to the STING transmembrane domain in the area of the hypothesized pore, making it a candidate for pharmacological inhibition of the proposed ion channel function of STING. Indeed, compared to treatment with diABZI alone, it was found that the STING-mediated Golgi pH increase was dramatically reduced upon treatment of cells with C53 along with diABZI, further emphasizing the requirement of the proposed pore region of STING for producing a change in the Golgi pH.

### Example 4: STING as a Channel for Proton Release

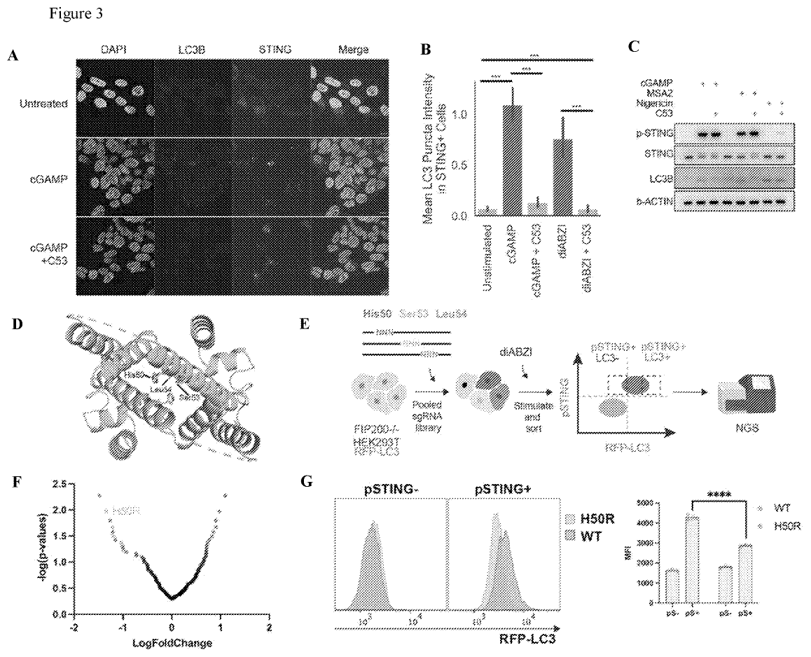

Given the impaired ion leakage upon co-treatment with C53, it was assessed whether STING-induced autophagy could also be inhibited by STING pore blockade via C53, which had not been previously tested for its effect on autophagy (Lu et al. 2022). Indeed, LC3 lipidation was induced upon treatment with both cGAMP and non-CDN agonists MSA2 or diABZI, while co-treatment with C53 strongly impaired LC3 lipidation despite no reduction in STING phosphorylation and similar levels of STING translocation (FIG. 3A-C). Notably, C53 co-treatment did not influence the induction of LC3 lipidation by Nigericin, an independent ionophore stimulus (FIG. 3C), demonstrating that the reduction in LC3 lipidation is mediated specifically by STING, rather than through downstream V-ATPase or other autophagy machinery.

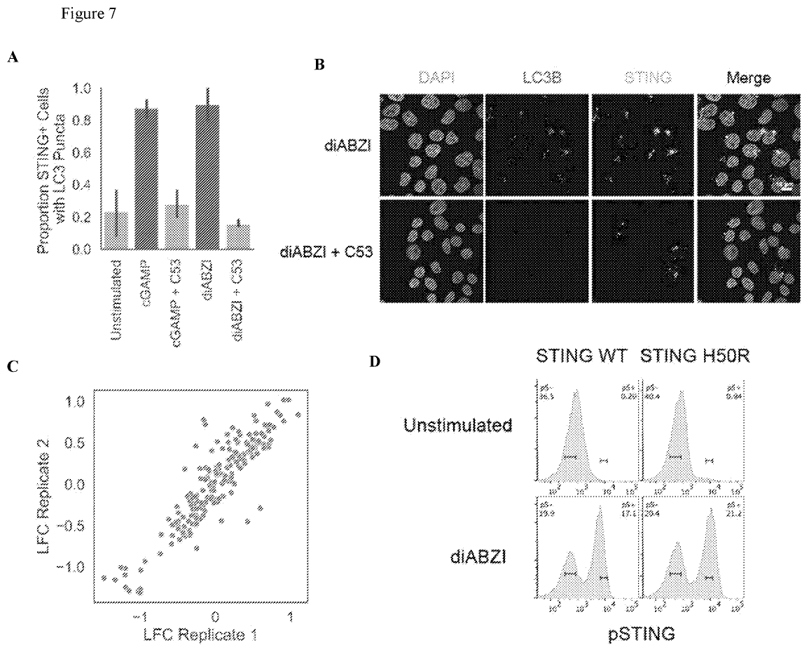

To further demonstrate that the proposed pore area is the mediator of STING-induced ion leakage, saturated mutagenesis of three critical amino acids (His50, Ser53, and Leu54 of human STING) whose side chains face the proposed pore area (FIG. 3d) was performed. A sorting strategy similar to the genome-wide CRISPR screen was followed, sorting populations of LC3− and LC3+ HEK293T cells within the pSTING+ cell population (FIG. 3E), thus ensuring that the sorted cell mutants enabled expression of functional STING able to translocate to Golgi/endosomes where phosphorylation of STING occurs (Dobbs et al. 2015). Among the top 12 mutants with reduced LC3 reduction upon agonist stimulation (FIG. 3F, Appendix I), 6 of them encoded the same amino acid change (H50R). The STING H50R mutant was therefore expressed in HEK293T cells and it was confirmed that LC3 induction within pSTING+ cells was reduced (FIG. 3G), despite having similar STING phosphorylation levels as the WT STING upon stimulation (FIG. 7B); consistent with a prior study showing that mutations in His50 do not reduce pSTING levels (Lu et al. 2022). The identification of a mutant that specifically blocks STING-induced autophagy without affecting STING phosphorylation, as seen through treatment with the small molecule C53, further supports that the proposed pore area is the key mediator of ion leak and STING-induced autophagy.

| Potential mutation | |
|---|---|
| sites in | |
| human STING | Relative location |
| Tyr46 | Transmembrane |
| | helix 2 (TM2) |
| His50 | TM2 |
| Ser53 | TM2 |
| Leu54 | TM2 |
| Ser127 | TM4 |
| Gln128 | TM4 |
| Asn131 | TM4 |
| Lys137 | TM4 |

| Human STING aa sequence | |
|---|---|
| (SEQ ID NO. 1) | |
| 1 | mphsslhpsi pcprghgaqk aalvllsacl vtlwglgepp ehtlrylvlh laslqlglll |
| 61 | ngvcslaeel rhihsryrgs ywrtvraclg cplrrgalll lsiyfyyslp navgppftwm |
| 121 | lallglsqal nillglkgla paeisavcek gnfnvahgla wsyyigylrl ilpelqarir |
| 181 | tynqhynnll rgavsqrlyi llpldcgvpd nlsmadpnir fldklpqqtg dhagikdrvy |
| 241 | snsiyellen gqragtcvle yatplqtlfa msqysqagfs redrleqakl fcrtledila |
| 301 | dapesqnncr liayqepadd ssfslsqevl rhlrqeekee vtvgslktsa vpststmsqe |
| 361 | pellisgmek plplrtdfs |

### Example 5: STING as a Channel for Proton Release

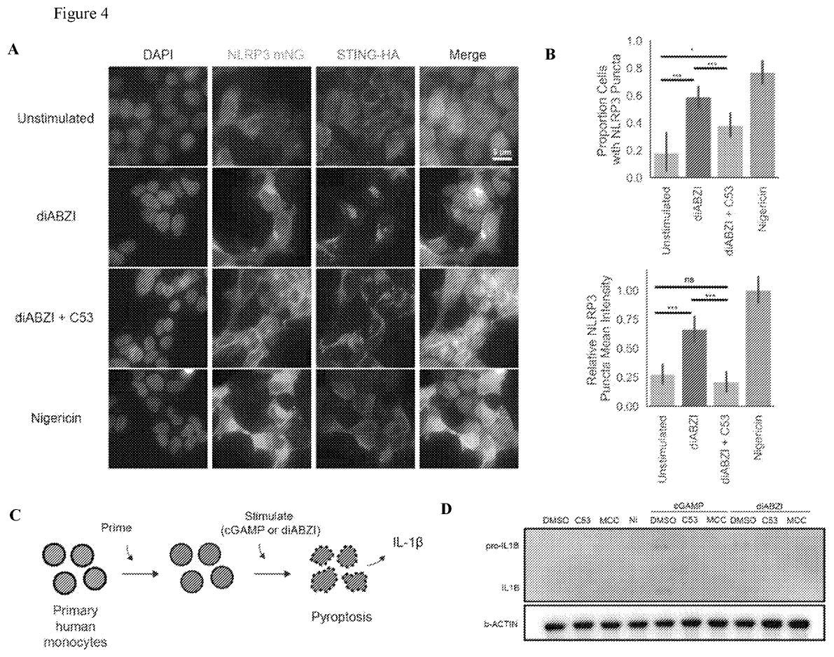

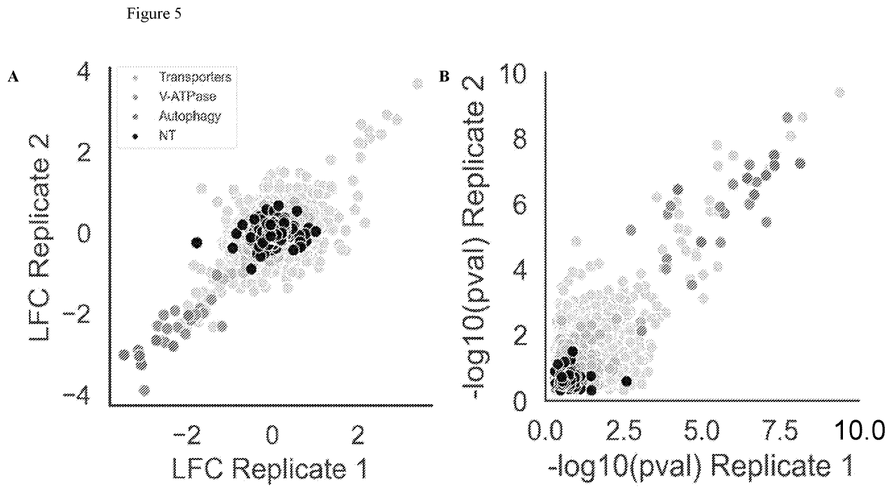

Besides autophagy, NLRP3-dependent inflammasome activation is another downstream effect of STING activation whose mechanism has not yet been fully elucidated (Gaidt et al. 2017). The previous finding that NLRP3 activation can be triggered by proton leakage through influenza M2 channel (Ichinohe, Pang, and Iwasaki 2010) may suggest that C53 would also inhibit STING-induced inflammasome activation. Another study showed that cytosolic NLRP3 translocates and concentrates on PI4P-enriched Golgi vesicles to initiate downstream inflammasome activation (Chen and Chen 2018). Given the finding disclosed herein that STING induces proton leakage from the Golgi, it was assessed whether STING-induced NLRP3 activation also requires ion leakage through the newly identified STING pore structure. Using an NLRP3-mNeongreen reporter system, it was found that NLRP3 translocated from the cytoplasm to perinuclear vesicles upon stimulation with STING agonist (FIG. 4A), similar to the effect induced by the known NLRP3 agonist nigericin (Chen and Chen 2018). In addition, it was found that NLRP3 colocalized strongly with STING on the perinuclear vesicles (FIG. 4A), suggesting that STING translocation to these vesicles caused the enrichment process and further supporting the role of STING-induced ion leakage as the key driver of NLRP3 activation. When cells treated with the STING agonist diABZI were co-treated with C53, a significant reduction in the proportion of cells with NLRP3 puncta and the mean NLRP3 puncta intensity was observed (FIG. 4A, B). Finally, inflammasome activation and subsequent IL-1ß release was tested in primary human monocytes induced by STING activation (FIG. 4C). Consistent with the NLRP3 phenotype, IL-1ß release induced by cGAMP or diABZI was also strongly reduced upon co-treatment with C53, similar to NLRP3 inhibitor MCC950, confirming that STING-induced inflammasome activation also depends on the channel function of STING (FIG. 4D).

### Example 6: Discussion

As disclosed herein, a pore in STING mediates ion leakage that is required for induction of non-canonical autophagy and NLRP3-dependent inflammasomes, as demonstrated by the combination of functional genomics, structural analysis, and chemical biology approaches. Observations were confirmed in multiple cell models, including hTERT-immortalized fibroblasts and primary human monocytes. The ion leakage that triggers autophagy is likely mediated by protons; however the effects on inflammasome activation are not as well understood and could be due to leakage of other ions. In addition to providing a mechanistic basis for STING-induced autophagy and inflammasome activation, the findings suggest that STING-induced ionic shifts may be capable of e.g., changing membrane voltage and impact immune cell and neuron activation.

### REFERENCES

Burdette, Dara L., Kathryn M. Monroe, Katia Sotelo-Troha, Jeff S. Iwig, Barbara Eckert, Mamoru Hyodo, Yoshihiro Hayakawa, and Russell E. Vance. 2011. “STING Is a Direct Innate Immune Sensor of Cyclic Di-GMP.” Nature 478 (7370): 515-18.
Chen, Jueqi, and Zhijian J. Chen. 2018. “PtdIns4P on Dispersed Trans-Golgi Network Mediates NLRP3 Inflammasome Activation.” Nature 564 (7734): 71-76.
Dobbs, Nicole, Nikolay Burnaevskiy, Didi Chen, Vijay K. Gonugunta, Neal M. Alto, and
Nan Yan. 2015. “STING Activation by Translocation from the ER Is Associated with Infection and Autoinflammatory Disease.” Cell Host & Microbe 18 (2): 157-68.
Feldman, David, Luke Funk, Anna Le, Rebecca J. Carlson, Michael D. Leiken, Funien Tsai, Brian Soong, Avtar Singh, and Paul C. Blainey. 2022. “Pooled Genetic Perturbation Screens with Image-Based Phenotypes.” Nature Protocols 17 (2): 476-512.
Fischer, Tara D., Chunxin Wang, Benjamin S. Padman, Michael Lazarou, and Richard J. Youle. 2020. “STING Induces LC3B Lipidation onto Single-Membrane Vesicles via the V-ATPase and ATG16L1-WD40 Domain.” The Journal of Cell Biology 219 (12). https://doi.org/10.1083/jcb.202009128.
Gaidt, Moritz M., Thomas S. Ebert, Dhruv Chauhan, Katharina Ramshorn, Francesca Pinci, Sarah Zuber, Fionan O'Duill, et al. 2017. “The DNA Inflammasome in Human Myeloid Cells Is Initiated by a STING-Cell Death Program Upstream of NLRP3.” Cell 171 (5): 1110-24.e18.
Gui, Xiang, Hui Yang, Tuo Li, Xiaojun Tan, Peiqing Shi, Minghao Li, Fenghe Du, and Zhijian J. Chen. 2019. “Autophagy Induction via STING Trafficking Is a Primordial Function of the cGAS Pathway.” Nature 567 (7747): 262-66.
Hooper, Kirsty M., Elise Jacquin, Taoyingnan Li, Jonathan M. Goodwin, John H. Brumell, Joanne Durgan, and Oliver Florey. 2022. “V-ATPase Is a Universal Regulator of LC3-Associated Phagocytosis and Non-Canonical Autophagy.” The Journal of Cell Biology 221 (6). https://doi.org/10.1083/jcb.202105112.
Ichinohe, Takeshi, Iris K. Pang, and Akiko Iwasaki. 2010. “Influenza Virus Activates Inflammasomes via Its Intracellular M2 Ion Channel.” Nature Immunology 11 (5): 404-10.
Ishikawa, Hiroki, and Glen N. Barber. 2008. “STING Is an Endoplasmic Reticulum Adaptor That Facilitates Innate Immune Signalling.” Nature 455 (7213): 674-78.
Linders, Peter T. A., Melina Ioannidis, Martin ter Beest, and Geert van den Bogaart. 2022. “Fluorescence Lifetime Imaging of pH along the Secretory Pathway.” ACS Chemical Biology 17 (1): 240-51.
Liu, Dong, Hao Wu, Chenguang Wang, Yanjun Li, Huabin Tian, Sami Siraj, Sheikh Arslan Sehgal, et al. 2019. “STING Directly Activates Autophagy to Tune the Innate Immune Response.” Cell Death and Differentiation 26 (9): 1735-49.
Lu, Defen, Guijun Shang, Jie Li, Yong Lu, Xiao-Chen Bai, and Xuewu Zhang. 2022. “Activation of STING by Targeting a Pocket in the Transmembrane Domain.” Nature 604 (7906): 557-62.
Pravda, Lukáš, David Sehnal, Dominik Toušek, Veronika Navrátilová, Václav Bazgier, Karel Berka, Radka Svobodová Vareková, Jaroslav Koca, and Michal Otyepka. 2018. “MOLEonline: A Web-Based Tool for Analyzing Channels, Tunnels and Pores (2018 Update).” Nucleic Acids Research 46 (W1): W368-73.
Sun, Lijun, Jiaxi Wu, Fenghe Du, Xiang Chen, and Zhijian J. Chen. 2013. “Cyclic GMP-AMP Synthase Is a Cytosolic DNA Sensor That Activates the Type I Interferon Pathway.” Science 339 (6121): 786-91.
Ulferts, Rachel, Elena Marcassa, Lewis Timimi, Liam Changwoo Lee, Andrew Daley, Beatriz Montaner, Suzanne Dawn Turner, Oliver Florey, John Kenneth Baillie, and Rupert Beale. 2021. “Subtractive CRISPR Screen Identifies the ATG16L1/vacuolar ATPase Axis as Required for Non-Canonical LC3 Lipidation.” Cell Reports 37 (4): 109899.
Wu, Jiaxi, Lijun Sun, Xiang Chen, Fenghe Du, Heping Shi, Chuo Chen, and Zhijian J. Chen. 2013. “Cyclic GMP-AMP Is an Endogenous Second Messenger in Innate Immune Signaling by Cytosolic DNA.” Science 339 (6121): 826-30.
Xu, Yue, Sen Cheng, Huan Zeng, Ping Zhou, Yan Ma, Lin Li, Xiaoyun Liu, Feng Shao, and Jingjin Ding. 2022. “ARF GTPases Activate Salmonella Effector SopF to ADP-Ribosylate Host V-ATPase and Inhibit Endomembrane Damage-Induced Autophagy.” Nature Structural & Molecular Biology 29 (1): 67-77.
Xu, Yue, Ping Zhou, Sen Cheng, Qiuhe Lu, Kathrin Nowak, Ann-Katrin Hopp, Lin Li, et al. 2019. “A Bacterial Effector Reveals the V-ATPase-ATG16L1 Axis That Initiates Xenophagy.” Cell 178 (3): 552-66.e20.
Zhang, Conggang, Guijun Shang, Xiang Gui, Xuewu Zhang, Xiao-Chen Bai, and Zhijian J. Chen. 2019. “Structural Basis of STING Binding with and Phosphorylation by TBK1.” Nature 567 (7748): 394-98.
Zhao, Baoyu, Fenglei Du, Pengbiao Xu, Chang Shu, Banumathi Sankaran, Samantha L. Bell, Mengmeng Liu, et al. 2019. “A Conserved PLPLRT/SD Motif of STING Mediates the Recruitment and Activation of TBK1.” Nature 569 (7758): 718-22.

### INCORPORATION BY REFERENCE

All publications and patents mentioned herein are hereby incorporated by reference in their entirety as if each individual publication or patent was specifically and individually indicated to be incorporated by reference. In case of conflict, the present specification, including its specific definitions, will control. While specific aspects of the patient matter have been discussed, the above specification is illustrative and not restrictive. Many variations will become apparent to those skilled in the art upon review of this specification and the claims below. The full scope should be determined by reference to the claims, along with their full scope of equivalents, and the specification, along with such variations.

### EQUIVALENTS

The present invention has been described in connection with what are presently considered to be the most practical and preferred embodiments. However, the invention has been presented by way of illustration and is not intended to be limited to the disclosed embodiments. Accordingly, one of skill in the art will realize that the invention is intended to encompass all modifications and alternative arrangements within the spirit and scope as set forth in the appended claims.

What is claimed is:

1. A mutant stimulator of interferon genes (STING) polypeptide comprising a mutation within its dimerization interface, wherein the mutation reduces the ability of the mutant STING polypeptide to mediate protein leakage across a membrane compared to a wild-type STING polypeptide of SEQ ID NO. 1.

2. The mutant STING polypeptide of claim 1, wherein the mutation is within a transmembrane helix of the mutant STING polypeptide.

3. The mutant STING polypeptide of claim 1, wherein the mutation is within TM2 or TM4.

4. The mutant STING polypeptide of claim 1, wherein the mutation is within TM2.

5. The mutant STING polypeptide of any one of claims 1-4, wherein the mutation is a substitution mutation.

6. The mutant STING polypeptide of any one of claims 1-5, wherein the mutation is located at a position of the mutant STING polypeptide corresponding to a position between amino acids Y46 and K137 of SEQ ID NO. 1.

7. The mutant STING polypeptide of claim 6, wherein mutation is located at a position of the mutant STING polypeptide corresponding to Y46, H50, S53, L54, S127, Q128, N131, or K137 of SEQ ID NO. 1.

8. The mutant STING polypeptide of claim 7, wherein the mutation is a H50R mutation.

9. A nucleic acid encoding the mutant STING polypeptide of any one of claims 1-8.

10. A vector comprising the nucleic acid of claim 9.

11. The vector of claim 10, wherein the vector is selected from nanoparticles, adenovirus vectors, adeno-associated virus (AAV) vectors, retrovirus vectors, picorna virus vectors, liposomes, cationic lipid systems, and protein/nucleic acid complexes.

12. A cell comprising the nucleic acid of claim 9.

13. A cell comprising the vector of claim 10.

14. A cell expressing the mutant STING polypeptide of any one of claims 1-8.

15. The cell of any one of claims 12-14, wherein the cell is an endothelial cell, epithelial cell, neuronal cell, or haematopoietic cell.

16. The cell of claim 15, wherein the haematopoietic cell is an immune cell selected from a lymphocyte, a monocyte, a macrophage, a dendritic cell, a mast cell, a neutrophil, a basophil, or an eosinophil.

17. The cell of claim 16, wherein the immune cell is a lymphocyte selected from a an αβT cell, γδT cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, or a regulatory T cell.

18. The cell of any one of claims 11-13, wherein the cell is a cell of the central nervous system (CNS) or peripheral nervous system (PNS).

19. The cell of any one of claims 12-14, wherein the cell is a cell of the bone marrow.

20. A nucleic acid of claim 9, wherein the nucleic acid is an mRNA.

21. A therapeutic composition comprising the mRNA of claim 20.

22. The therapeutic composition of claim 21, comprising a vector selected from nanoparticles, adenovirus vectors, adeno-associated virus (AAV) vectors, retrovirus vectors, picorna virus vectors, liposomes, cationic lipid systems, and protein/nucleic acid complexes.

23. A therapeutic composition comprising an mRNA of claim 20 formulated in a lipid nanoparticle (LNP).

24. The therapeutic composition of any one of claims 21 to 23, wherein one or more of the uridine nucleosides in the mRNA are N1-methylpseudouridine.

25. The therapeutic composition of claim 24, wherein all of the uridine nucleosides in the mRNA are N1-methylpseudouridine.

26. The therapeutic composition of any one of claims 21-25, wherein the LNP comprises an ionizable lipid, a structural lipid, a phospholipid, a sterol, a PEG-modified lipid, or any combination thereof.

27. A method of treating a cancer in a subject, the method comprising administering an effective amount of the therapeutic composition of any one of claims 21-26.

28. The method of claim 27, wherein the cancer is selected from: hepatocellular carcinoma, lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

29. The method of claim 27 or 28, wherein the therapeutic composition is administered intrapleurally, intravenously, subcutaneously, intranodally, intratumorally, intrathecally, intraperitoneally, intracranially, or by direct administration to an organ.

30. The method of any one of claims 27-29, further comprising administering to the subject a cancer immunotherapy.

31. The method of claim 30, wherein the cancer immunotherapy comprises administration of an immune checkpoint inhibitor.

32. The method of claim 31, wherein the immune checkpoint inhibitor comprises an antibody or antigen-binding fragment thereof specific for PD-1, PD-L1, or CTLA4.

33. The method of claim 32, wherein the cancer immunotherapy comprises administration of an engineered T cell, CAR-T cell or a CAR-NK cell.
